(19) United States
(12) Patent Application Publication
(10) Pub. No.: US 2022/0226661 A1
(43) Pub. Date: Jul. 21, 2022

(54) ELECTROMAGNETIC DEVICE AND METHOD FOR TREATING CANCERS AND TUMORS

(71) Applicant: LEV P. DVORKIN, K. BIALEK (IL)

(72) Inventor: LEV P. DVORKIN, K. BIALEK (IL)

(21) Appl. No.: 17/521,337

(22) Filed: Nov. 8, 2021

Related U.S. Application Data
(60) Provisional application No. 63/140,114, filed on Jan. 21, 2021.

(51) Int. Cl. A61N 1/40

(52) U.S. Cl. CPC A61N 1/40

(57) ABSTRACT

The electromagnetic device and method for treating cancers or tumors changes the metabolism of cells, helping to stop proliferation of cancers or tumor cells inside or on the body. The device includes an RF frequency generator for generating an RF carrier signal, a low frequency generator for generating a modulating signal, a modulator for frequency modulating the RF carrier signal with the modulating signal to produce a frequency-modulated output signal, and an antenna with a parabolic trough reflector for directing the modulated signal to a patient's body. The method includes applying the signal for predetermined periods of time with a power density of between 1 mW/cm² and 15 mW/cm² at an RF frequency between 100 MHz and 900 MHz, more specifically between 400 MHz and 500 MHz, the carrier signal being frequency-modulated by a modulating signal between 100 Hz and 10 kHz, more specifically 1-4 kHz.

### CROSS-REFERENCE TO RELATED APPLICATIONS

This application claims the benefit of U.S. Provisional Patent Application No.

63/140,114, filed on Jan. 21, 2021.

### BACKGROUND

### 1. Field

The disclosure of the present patent application relates to RF therapy devices, and particularly to an electromagnetic device and method for treating cancers and tumors.

### 2. Description of the Related Art

According to the World Health Organization, a study in 2018 indicated that every fifth man and every sixth woman will get cancer at some stage of their life. The annual cost of treating skin cancers in the U.S. is estimated at $8.1 billion, including about $4.8 billion for non-melanoma skin cancers, and $3.3 billion for melanoma. In the U.S., more than 9,500 people are diagnosed with skin cancer every day. More than two people die of the disease every hour. Basal cell carcinoma (BCC) is the most common form of skin cancer. An estimated 4.3 million cases of BCC are diagnosed in the U.S. each year. Squamous cell carcinoma (SCC) is the second most common form of skin cancer. More than 1 million cases of SCC are diagnosed in the U.S. each year. Organ transplant patients are approximately 100 times more likely than the general public to develop squamous cell carcinoma. Current figures suggest that more than 15,000 people die of SCC in the U.S. each year, more than twice as many as from melanoma. Neuroblastoma is by far the most common cancer in infants (younger than 1 year old). There are about 700 to 800 new cases of neuroblastoma each year in the United States. This number has remained about the same for many years. The National Institutes of Health (NIH) estimates that 80% of all women will develop uterine fibroids (myomas) at some point during their lives. Because many women don't experience any symptoms, it's possible that the incidence of uterine fibroids is even higher. Fibroids are considered benign or noncancerous, but can be painful.

Some current RF therapy devices for treating cancer use amplitude-modulated RF signals. While these devices are somewhat effective, they are limited in their performance, and their ability to treat neuroblastoma, squamous cell carcinoma, and benign (noncancerous) tumors, such as myoma, has not been thoroughly evaluated.

Thus, an electromagnetic device and method for treating cancers and tumors solving the aforementioned problems is desired.

### SUMMARY

The electromagnetic device and method for treating cancers and tumors changes the metabolism of cells, helping to stop proliferation of tumors and cancer cells inside or on the body and providing epigenetic reprogramming of cancer cells into regular-like cells for a complete cancer cure. The device includes an RF frequency generator for generating an RF signal, a low frequency generator for generating a modulating signal, a modulator for frequency modulating the RF signal with the modulating signal to produce a frequency-modulated output signal, and an antenna with a parabolic trough reflector (PTR) for directing the modulated signal to a patient's body.

The method includes applying the signal for predetermined periods of time at a power density of between 1 mW/cm² and 15 mW/cm², more specifically between 10 mW/cm² and 15 mW/cm², and at an RF frequency between 100 MHz and 900 MHz, more specifically between 400 MHz and 500 MHz and at a modulation frequency between 100 Hz and 10 kHz, more specifically 1-4 kHz, which allows maintaining the mode of slow metabolism for thirty minutes. In one embodiment of the method, a 10 W signal with an RF carrier frequency of 430 MHz and modulation frequency of 1000 Hz reduced the temperature in cancer cells by 2.5° C. during irradiation, with up to 80% reduction of cancer cells growth and reprogramming of the cancer cells. Cancers that can be treated using the electromagnetic device and method include, but are not limited to, neuroblastoma, glioblastoma, and squamous cell carcinoma (SCC). In addition, benign (noncancerous) tumors, such as myoma, can also be treated using the electromagnetic device and method.

The method can be repeated for several hours over several days to achieve 100% inhibition of cancer cell growth and to finish reprogramming of all cancer cells in order to stop cancer cell proliferation of any kind. The number of procedures to achieve reprogramming of all cancer cells is dependent on the speed of proliferation of the specific cancer or tumor type. Generally, slow proliferation cancers require more irradiation procedures and more days of treatment in comparison with fast proliferation cancers.

These and other features of the present subject matter will become readily apparent upon further review of the following specification.

### BRIEF DESCRIPTION OF THE DRAWINGS

FIG. 1 is a perspective view of an electromagnetic (EM) device for treating cancers and tumors.

FIG. 2 is a schematic diagram of the of the EM device for treating cancers and tumors of FIG. 1.

FIG. 3 is a graph showing changes in the temperature of Sansevieria plant leaves as a function of time for exemplary modulation frequencies induced by the EM device for treating cancers and tumors of FIG. 1.

FIG. 4 is a plot showing changes in the temperature of SCC Cancer Cells as a function of time during irradiation by frequency-modulated RF electromagnetic waves generated by the EM device for treating cancers and tumors of FIG. 1.

FIG. 5A is a chart showing inhibition of neuroblastoma cancer cell (SHSY5Y) growth after irradiation by a frequency-modulated RF EM field generated by the EM device for treating cancers and tumors of FIG. 1.

FIG. 5B is a chart showing inhibition of SCC cancer cell (SCC47) growth after irradiation by a frequency-modulated RF EM field generated by the EM device for treating cancers and tumors of FIG. 1.

FIG. 6 is a plot of temperature changes of SCC cancer cells as a function of time, comparing temperature change during initial exposure to temperature change during a second exposure three hours later to a frequency-modulated RF EM field generated by the EM device for treating cancers and tumors of FIG. 1.

FIG. 7A is a chart showing inhibition of SCC cancer cell SCC47 growth after exposure to various frequency-modulated RF EM fields generated by the EM device for treating cancers and tumors of FIG. 1.

FIG. 7B is a chart showing inhibition of neuroblastoma cancer cell SHSY5Y growth after exposure to various frequency-modulated RF EM fields generated by the EM device for treating cancers and tumors of FIG. 1.

FIG. 8A is a chart showing inhibition of SCC cancer cell SCC47 cell growth after exposure to various frequency-modulated RF EM fields generated by the EM device for treating cancers and tumors of FIG. 1.

FIG. 8B is a chart showing inhibition of SCC cancer cell SCC47 and neuroblastoma cancer cell SHSY5Y growth after exposure to various frequency-modulated RF EM fields generated by the EM device for treating cancers and tumors of FIG. 1.

FIG. 9 is a graph of temperature changes of Sansevieria plant leaves as a function of power density of a frequency-modulated RF EM field generated by the EM device for treating cancers and tumors of FIG. 1.

Similar reference characters denote corresponding features consistently throughout the attached drawings.

### DETAILED DESCRIPTION OF THE PREFERRED EMBODIMENTS

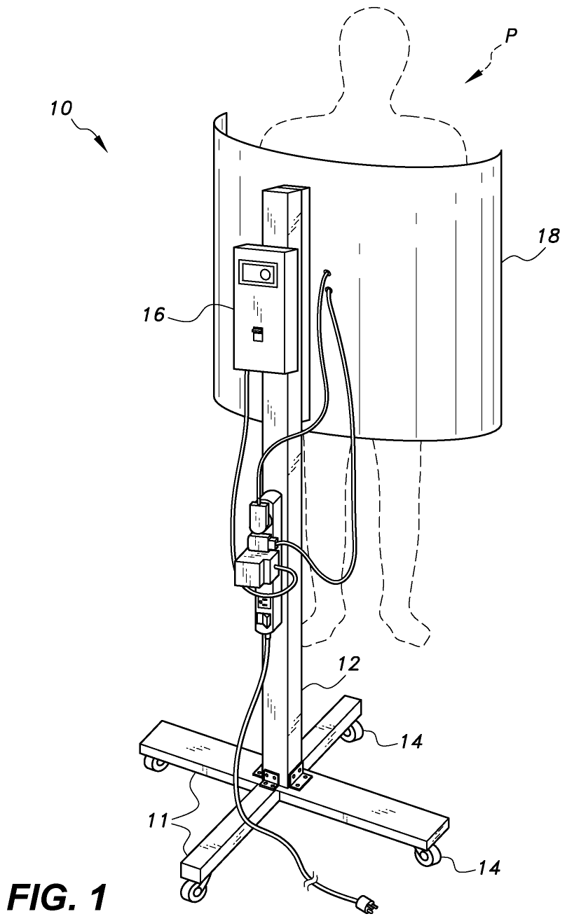

An exemplary electromagnetic (EM) device 10 for treating cancers and tumors is shown in FIG. 1. The device 10 includes a support holder or stand having a generally vertical support column 12 extending upward from the proximate end of four horizontal lower support members 11. The distal end of the horizontal lower support members 11 may include caster-type wheels 14 to allow the device 10 to be rolled into position for use or storage. The generally vertical support column 12 and the horizontal lower support members 11 may be made of aluminum. It should be noted that the holder for the device 10 may be of any suitable configuration, and the floor stand shown in FIG. 1 is for illustrative purposes only. The upper end of the vertical support column 12 has an electronic control box 16 and a parabolic trough reflector 18 mounted on opposite sides thereof. In use, a patient P is positioned at predetermined distance in front of the parabolic trough reflector 18 for receiving the RF energy from the device 10. For example, the predetermined distance may be between 0.5 m to 2 m.

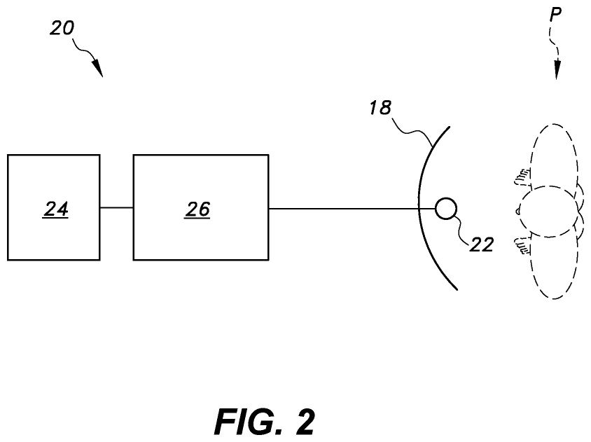

FIG. 2 shows a schematic diagram 20 of the EM device 10 of FIG. 1. The device 10 includes a low frequency generator 24 that provides a modulating signal of between 100 Hz to 10 kHz. The modulating signal is fed to block 26, which includes an FM modulator and a high frequency generator that provides an RF carrier signal in portions of the very high frequency (VHF) and ultra-high frequency (UHF) RF bands between 100 MHz and 900 MHz. The resulting FM RF signal is provided to an antenna element 22 at a power density of between 1 mW/cm² and 15 mW/cm² and is directed toward the patient P using the parabolic trough reflector 18. In addition to the above-described circuitry, the EM device 10 also includes one or more power supplies (not shown) for providing the required voltages for the various circuits, as is known in the art.

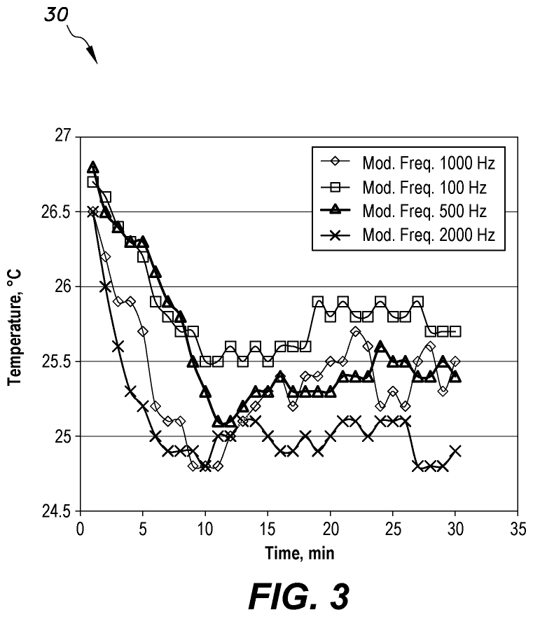

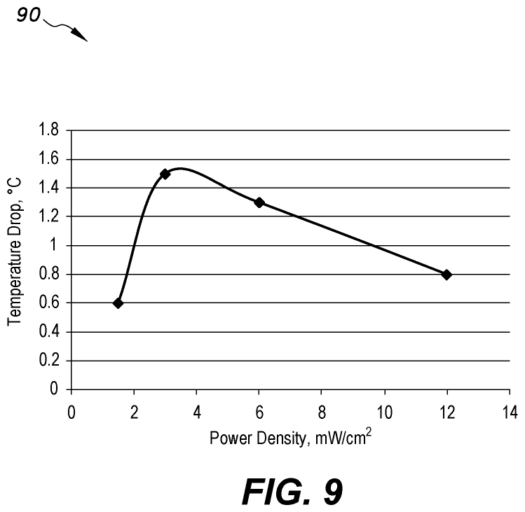

FIG. 3 is a graph 30 showing changes in the temperature of Sansevieria plant leaves for modulation frequencies of 100 Hz, 500 Hz, 1000 Hz and 2000 Hz of the FM RF EM waves from the EM device 10, the temperature being related to the metabolism of the Sansevieria plant leaves. It can be seen that for all of the modulation frequencies the plant temperature reduces over time due to exposure to the FM RF EM waves. FIG. 9 is a graph 90 of temperature change (and change in metabolism) of Sansevieria plant leaves as a function of power density of a frequency-modulated RF EM field generated by the EM device 10. The graph indicates that from a minimal power density of 1.5 mW/cm² to a power density of 3.5 mW/cm² there is an increasing amount of temperature drop of the Sansevieria plant leaves to a maximum temperature drop of 1.5° C. at a power density of 3.5 mW/cm². At power densities above 3.5 mW/cm² the temperature drop decreases in a linear manner. While not being bound by theory, this effect can be explained by considering two different physical processes. The first process works at low RF power density, when electrical forces induced by virtual photons interact with electrical fields of single nucleotides (where each single nucleotide is an electrical dipole) and this interaction decreases the Sansevieria plant cell temperature and metabolism. The second process starts to have a noticeable effect at higher power densities, when a large number of real photons results in partial absorption of these photons and their energy, and as result, above 3.5 mW/cm² the second process starts to offset the first process. With further increasing of the RF power density, the Sansevieria plant cell temperature will continue to increase until the temperature drop is a temperature rise.

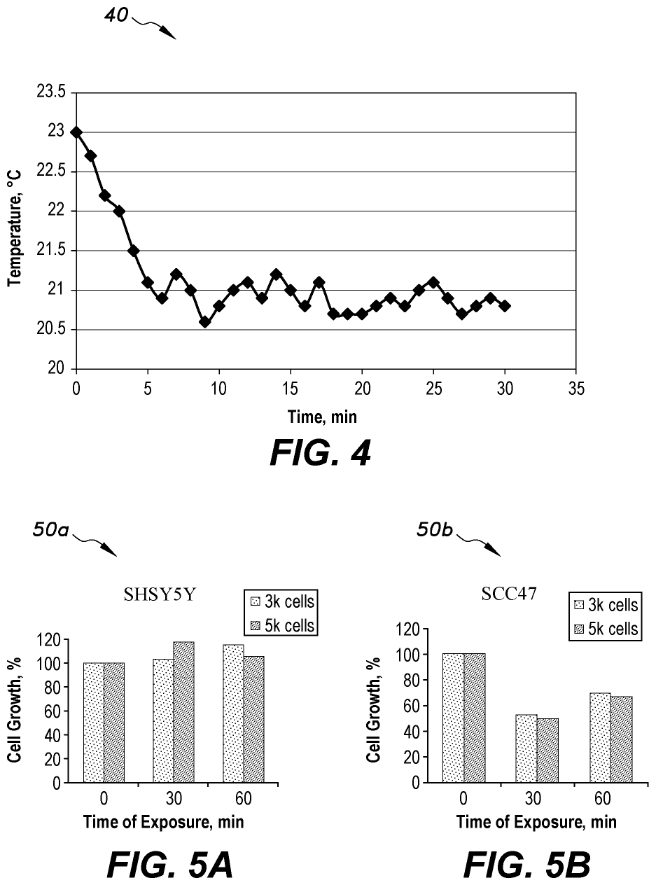

FIG. 4 is a plot 40 showing changes in the temperature of SCC cancer cells induced by FM RF EM waves from the EM device 10. The decreasing temperature of the SCC cancer cells over time indicates changes in the cells' metabolism during irradiation by the FM RF EM waves.

FIG. 5A is a chart 50a showing inhibition of neuroblastoma cancer cells (SHSY5Y) growth after exposure to an FM RF EM field generated by the EM device 10.

FIG. 5B is a chart 50b showing inhibition of SCC cancer cells (SCC47) growth after exposure to an FM RF EM field generated by the EM device 10.

FIG. 5B shows clear inhibition of the SCC cancer cells growth after irradiation, which indicates the reprogramming of the SCC cancer cells.

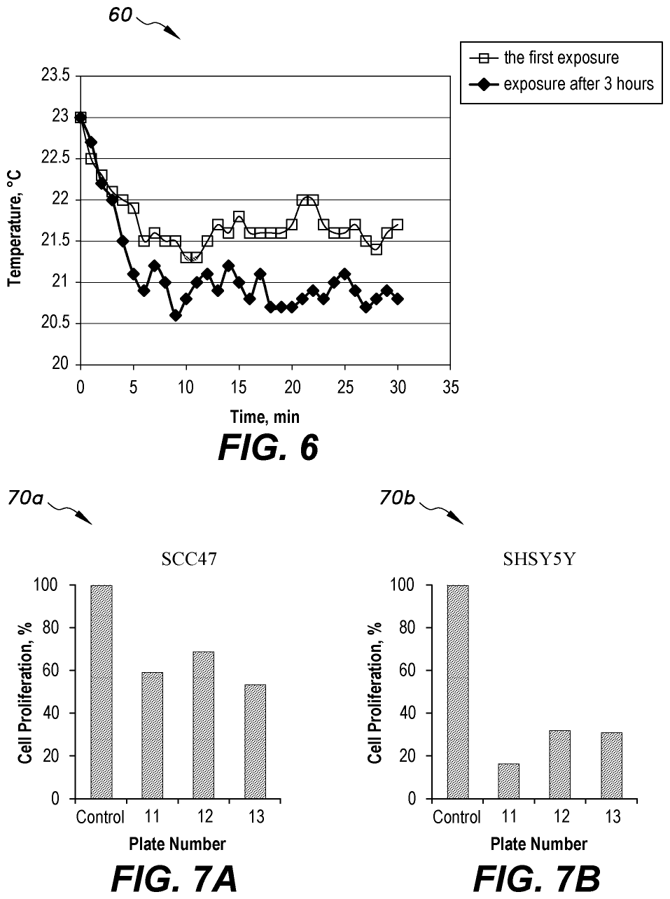

FIG. 6 is a plot 60 showing temperature changes of SCC cancer cells as a function of time during an initial 30-minute exposure to an FM RF EM field generated by the EM device 10 and during a second 30-minute exposure three hours later. The decreasing temperature of the SCC cancer cells over time indicates changes in the cells' metabolism during irradiation by the FM RF EM waves.

FIG. 7A is a chart 70a showing inhibition of SCC cancer cell (SCC47) growth after exposure to various frequency modulated RF EM fields from the EM device 10. On the x-axis, the control plate is for a sample that was not irradiated. Plate 11 indicates the reduced cell proliferation after a single 30-minute exposure to a 430 MHz RF carrier frequency modulated with a 1000 Hz modulating signal. Plate 12 indicates the reduced cell proliferation after a thirty-minute exposure including a first 10-minute exposure to a 430 MHz RF field frequency modulated with a 1000 Hz modulating signal, followed by a second 10-minute exposure to a 430 MHz RF field frequency modulated with a 500 Hz modulating signal, followed by a third 10-minute exposure to a 430 MHz RF field frequency modulated with a 200 Hz modulating signal. Plate 13 indicates the reduced cell proliferation after a first 30-minute exposure to a 430 MHz RF carrier frequency modulated with a 1000 Hz modulating signal, followed by a three-hour break between exposures, followed by a second 30-minute exposure to a 430 MHz RF carrier frequency modulated with a 500 Hz modulating signal.

FIG. 7B is a chart 70b showing inhibition of neuroblastoma cancer cell (SHSY5Y) growth after exposure to various frequency modulated RF EM fields generated by the EM device 10. On the x-axis, the plate designations are the same as in chart 70a of FIG. 7A.

FIGS. 7A and 7B show inhibition in the growth of cancer cells SCC47 (squamous cell carcinoma) and cancer cells SHSY5Y (neuroblastoma), which indicates a change in the cells' metabolism during irradiation using several different modulating frequencies and durations of FM RF EM fields generated by the EM device 10. From a comparison of charts 70a and 70b, it can be seen that there is a difference in reprogramming process efficiency for the two different kinds of the cancer cells (SCC47 and SHSY5Y) after the same irradiation time. This can be attributed to the proliferation speed of the SCC47 cells, which, in this case, was half the proliferation speed of the SHSY5Y cells.

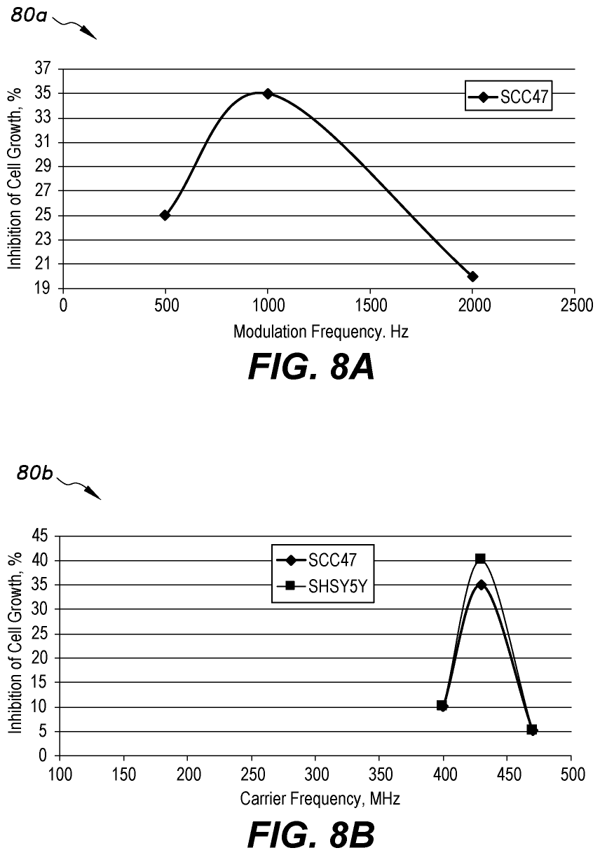

FIG. 8A is a chart 80a showing inhibition of SCC cancer cell SCC47 cell growth after exposure to various frequency-modulated RF EM fields generated by the EM device 10 with a modulation frequency from 500-2000 Hz. As can be seen from the chart 80a, the inhibition of the SCC cancer cells peaks at 35% with a 1000 Hz modulation frequency.

FIG. 8B is a chart 80b showing inhibition of SCC cancer cell SCC47 and neuroblastoma cancer cell SHSY5Y growth after exposure to various frequency-modulated RF EM fields generated by the EM device 10 with a carrier frequency from 400-470 Hz. As can be seen from the chart 80b, the inhibition of both types of cancer cells peaks at about a 430 Hz carrier frequency. As can also be seen from the chart 80b, the inhibition of the SCC cancer cells is 5% less than the inhibition of the neuroblastoma cancer cell at a 430 Hz carrier frequency. As previously noted, this can be attributed to the proliferation speed of the SCC47 cells, which in this case was half the proliferation speed of the SHSY5Y cells.

As previously noted, slow proliferation cancers require more irradiation procedures and more days of treatment in comparison with fast proliferation cancers. In order to increase the reprogramming efficiency to 100%, the irradiation process should be repeated several times in a 48-to-72-hour period. From testing, it has been found that the optimal number of 30-minute procedures for squamous cell carcinoma SCC47 cancer is twice a day with a 3-hour delay between procedures and requires repeating these procedures for four days. For neuroblastoma cancer cell SHSY5Y cancer, it was found that one 30-minute procedure per day for three days was adequate. The required number and schedule of irradiation procedures for maximum process efficacy can be calculated from cancer cells proliferation speed and can be confirmed experimentally.

It is to be understood that the electromagnetic device and method for treating cancers and tumors is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. An electromagnetic device for treating cancers and tumors, comprising:
a low frequency generator configured for providing a modulating signal;
a radio frequency (RF) frequency generator configured for providing an RF carrier signal;
a frequency modulator connected to the low frequency generator and to the RF frequency generator, the frequency modulator being configured for frequency modulating the RF carrier signal with the modulating signal;
an antenna for transmitting the frequency-modulated RF carrier signal; and
a reflector connected to the antenna for directing the transmitted frequency modulated RF carrier signal toward a patient's body.

2. The electromagnetic device of claim 1, wherein the reflector is a parabolic trough reflector.

3. A method for treating cancers, comprising the step of transmitting a frequency-modulated radio frequency (RF) electromagnetic signal directed at a site of a cancer in a patient afflicted with the cancer.

4. The method for treating cancers according to claim 3, wherein the step of transmitting a frequency-modulated RF electromagnetic signal further comprises transmitting the frequency-modulated RF electromagnetic signal from a distance between 0.5 m and 2 m from the site of the cancer.

5. The method for treating cancers according to claim 3, wherein the step of transmitting the frequency-modulated RF electromagnetic signal further comprises transmitting the frequency-modulated RF signal at a power density of between 1 mW/cm² and 15 mW/cm².

6. The method for treating cancers according to claim 3, wherein the step of transmitting the frequency-modulated RF electromagnetic signal further comprises transmitting a frequency-modulated RF signal having a carrier frequency of between 100 MHz and 900 MHz.

7. The method for treating cancers according to claim 6, wherein the carrier frequency of the transmitted frequency-modulated RF electromagnetic signal is between 400 MHz and 500 MHz.

8. The method for treating cancers according to claim 7, wherein the carrier frequency of the transmitted frequency-modulated RF electromagnetic signal is 430 MHz.

9. The method for treating cancers according to claim 6, further comprising the step of frequency modulating the transmitted RF signal with a modulating signal having a frequency between 100 Hz and 10 kHz.

10. The method for treating cancers according to claim 3, wherein the step of transmitting a frequency-modulated radio frequency (RF) electromagnetic signal comprises transmitting the signal for a plurality of transmitting procedures a day on at least one day.

11. The method for treating cancers according to claim 10, wherein:
the cancer is squamous cell carcinoma; and
the step of transmitting a frequency-modulated radio frequency (RF) electromagnetic signal comprises transmitting the signal for two 30-minute transmitting procedures with a 3-hour delay between the transmitting procedures on each of four consecutive days.

12. The method for treating cancers according to claim 10, wherein:
the cancer is neuroblastoma; and
the step of transmitting a frequency-modulated radio frequency (RF) electromagnetic signal comprises transmitting the signal for one 30-minute transmitting procedure on each of three consecutive days.

13. A method for treating tumors, comprising the step of transmitting a frequency-modulated radio frequency (RF) electromagnetic signal directed at a site of a tumor in a patient afflicted with the tumor.

14. The method for treating tumors according to claim 13, wherein the step of transmitting a frequency-modulated RF electromagnetic signal further comprises transmitting the frequency-modulated RF electromagnetic signal from a distance between 0.5 m and 2 m from the site of the tumor.

15. The method for treating tumors according to claim 13, wherein the step of transmitting the frequency-modulated RF electromagnetic signal further comprises transmitting the frequency-modulated RF signal at a power density of between 1 mW/cm² and 15 mW/cm².

16. The method for treating tumors according to claim 13, wherein the step of transmitting the frequency-modulated RF electromagnetic signal further comprises transmitting a frequency-modulated RF signal having a carrier frequency of between 100 MHz and 900 MHz.

17. The method for treating tumors according to claim 16, wherein the carrier frequency is between 400 MHz and 500 MHz.

18. The method for treating tumors according to claim 17, wherein the carrier frequency is 430 MHz.

19. The method for treating tumors according to claim 16, further comprising the step of frequency modulating the transmitted RF signal with a modulating signal having a frequency between 100 Hz and 10 kHz.

20. The method for treating tumors according to claim 13, wherein:
the tumor is myoma; and
the step of transmitting a frequency-modulated radio frequency (RF) electromagnetic signal comprises transmitting the signal for a plurality of transmitting procedures on at least one day.
